(19) United States
(12) Patent Application Publication
(10) Pub. No.: US 2021/0038335 A1
(43) Pub. Date: Feb. 11, 2021

(54) Robot Assisted Surgical System with Clutch Assistance

(71) Applicant: TransEnterix Surgical, Inc., Morrisville, NC (US)

(72) Inventor: Alexander John Maret, Apex, NC (US)

(21) Appl. No.: 16/931,426

(22) Filed: Jul. 16, 2020

Related U.S. Application Data
(60) Provisional application No. 62/874,973, filed on Jul. 16, 2019.

(51) Int. Cl. A61B 34/00; A61B 34/37

(52) U.S. Cl. CPC A61B 34/77; A61B 34/37; A61B 2017/00119; A61B 34/76; A61B 34/25; A61B 34/74

(57) ABSTRACT

A robot-assisted surgical system includes a robotic manipulator for robotic positioning of a surgical instrument, and a user input device moveable by a user to cause the robotic manipulator to move the surgical instrument. The system is configured to define virtual boundaries in a workspace of the user input device, based on range limits or user ergonomic limits of the user input device. The system alerts the user if the user input device is moved into proximity of the virtual boundary. This cues the user that it would be useful to clutch and reposition the user input device.

This application claims the benefit of US Provisional Application No. 62/874,973, filed Jul. 16, 2019.

### BACKGROUND

Surgical robotic systems are typically comprised of one or more robotic manipulators and a user interface. The robotic manipulators carry surgical instruments or devices used for the surgical procedure. A typical user interface includes input devices, or handles, manually moveable by the surgeon to control movement of the surgical instruments carried by the robotic manipulators. The surgeon uses the interface to provide inputs into the system and the system processes that information to develop output commands for the robotic manipulator. The user interface is designed to enable a more ergonomic positioning of the user's hands and arms. This means that the position and orientation of the user's hands and arms is no longer deterministic to the position of the surgical instrument end effector. In breaking this link between end effector and user interface, the surgeon can position the handles in an orientation that is more comfortable for the surgeon compared with the instrument handle positions during manual laparoscopic surgery. This helps to minimize the physical fatigue often associated with laparoscopic procedures. The user can maximize the ergonomics of the interface by “clutching,” which means temporarily disabling output motion at the surgical instrument in response to movement of the input device, to allow the surgeon to move the input device to a position that allows the surgeon to more comfortably manipulate the handle.

Another feature of physically separating the handle from the surgical instrument's end effector is that motion scaling is possible. This means that the user can adjust the relative amount of motion between the input and the output. If the user would like to create more precise motions at the instrument end effector, s/he can scale the end effector motion relative to the handle motion such that greater handle motion is required per unit of end effector motion. In this scenario, however, the user interface may have range of motion limitations where laparoscopic instruments did not. Once the surgeon has reached a range of motion limitation, s/he must “clutch out” in order to reposition the user interface prior to “clutching in” and regaining control of the instrument end effector.

Some systems may include predetermined or user-selectable motion scaling, in which a scaling factor is applied between the velocity of motion of the user input given at the input devices and the resulting velocity at which the corresponding robotic manipulator moves the surgical instrument. Surgeons may desire a fine scaling motion for certain procedures or steps, while in others s/he may prefer larger motion, relative to the movement of the user interface.

Some systems are configured to communicate to the surgeon the forces that are being applied to the patient by the surgical devices moved by the robotic manipulators. Communication of information representing such forces to the surgeon via the surgeon interface is referred to as “tactile feedback” or “haptic feedback.” In systems such as the one described in application US 2013/0012930, tactile feedback is communicated to the surgeon in the form of forces applied by motors to the surgeon interface, so that as the surgeon moves the handles of the surgeon interface, s/he feels resistance against movement representing the direction and magnitude of forces experienced by the robotically controlled surgical device. In some systems, motors at the surgeon interface are also used to perform active gravity compensation at the user input devices.

Co-pending and commonly owned U.S. application Ser. No. ______, entitled Auto Home Zone and Slow Correction for Robotic Surgical System User interface, filed Jul. 16, 2020, which is incorporated herein by reference, describes a system and method that assists the surgeon in positioning of the user input device to maximize its range of motion, thus minimizing the impact and frustration of reaching range of motion limitations during use. It does so by controlling motors at the surgeon interface, such as those used to generate haptic feedback, to apply forces to the user input to cause movement of the user input to a predetermined position or region.

This application describes concepts intended to improve the usability of the robotic system by enabling the user to more naturally and quickly reposition his/her hands near the ends of the range of motion of the input device or when entering an uncomfortable ergonomic position. These concepts will also improve the comfort of the users by encouraging clutching and repositioning which may reduce injuries related to the use of our device and increase the utilization of the device and length of surgeon careers via improved ergonomics. With some existing systems, users sometimes feel that they have to perform a significant amount of clutching during the procedure. This is an especially noticeable problem when using low motion scaling settings. When the user reaches the position limits of the input device or enters an uncomfortable ergonomic position, he/she can activate or release a button, pedal, trigger, presence sensor, etc to disable the motion of the output device and move to a more comfortable pose before re-enabling the output device. The disclosed concepts aim to increase the usability of this clutching process using haptic constraints, force gestures, and/or haptic feedback.

### BRIEF DESCRIPTION OF THE DRAWINGS

FIG. 1 shows an example of a robot-assisted surgical system;

FIG. 2 is a functional block diagram illustrating features of a first method according to the disclosed principles.

FIG. 3 is a functional block diagram illustrating features of a second method according to the disclosed principles.

FIG. 4 is a functional block diagram illustrating features of a third method according to the disclosed principles.

### DETAILED DESCRIPTION

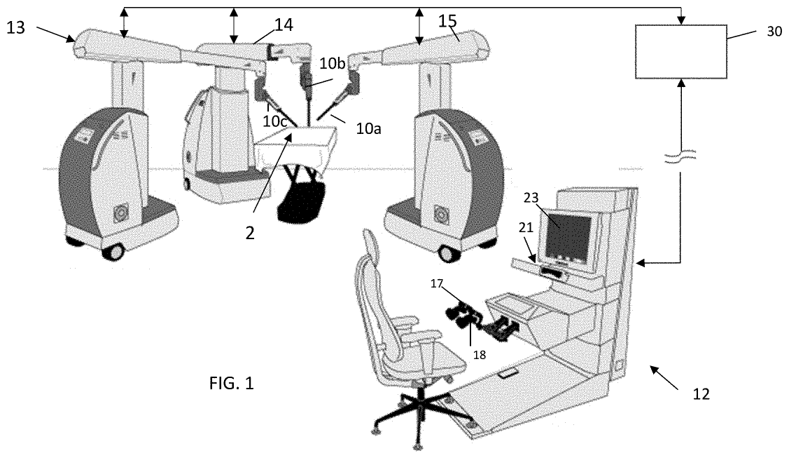

Although the inventions described herein may be used on a variety of robotic surgical systems, the embodiments will be described with reference to a system of the type shown in FIG. 1. In the illustrated system, a surgeon console 12 has two input devices such as handles 17, 18. The input devices 12 are configured to be manipulated by a user to generate signals that are used to command motion of a robotically controlled device in multiple degrees of freedom. In use, the user selectively assigns the two handles 17, 18 to two of the robotic manipulators 13, 14, 15, allowing surgeon control of two of the surgical instruments 10a, 10b, and 10c disposed at the working site at any given time. To control a third one of the instruments disposed at the working site, one of the two handles 17, 18 is operatively disengaged from one of the initial two instruments and then operatively paired with the third instrument. A fourth robotic manipulator, not shown in FIG. 1, may be optionally provided to support and maneuver an additional instrument.

One of the instruments 10a, 10b, 10c is a camera that captures images of the operative field in the body cavity. The camera may be moved by its corresponding robotic manipulator using input from a variety of types of input devices, including, without limitation, one of the handles 17, 18, additional controls on the console, a foot pedal, an eye tracker 21, voice controller, etc. The console may also include a display or monitor 23 configured to display the images captured by the camera, and for optionally displaying system information, patient information, etc.

A control unit 30 is operationally connected to the robotic arms and to the user interface. The control unit receives user input from the input devices corresponding to the desired movement of the surgical instruments, and the robotic arms are caused to manipulate the surgical instruments accordingly.

The input devices 17, 18 are configured to be manipulated by a user to generate signals that are processed by the system to generate instructions used to command motion of the manipulators in order to move the instruments in multiple degrees of freedom.

One or more of the degrees of freedom of the input devices are coupled with an electromechanical system capable of providing tactile haptic feedback to the surgeon, and optionally providing gravity compensation for the user input, and/or. It should be understood that the concepts described in this application are not limited to any particular user input device configuration. Alternative configurations include, without limitation, those described in co-pending application Ser. No. 16/513,670, entitled HAPTIC USER INTERFACE FOR ROBOTICALLY CONTROLLED SURGICAL INSTRUMENTS (Atty Ref: TRX-10610, attached at the Appendix), and user interfaces or haptic devices known to those of skill in the art or developed in the future.

The surgical system allows the operating room staff to remove and replace surgical instruments carried by the robotic manipulator, based on the surgical need. Once instruments have been installed on the manipulators, the surgeon moves the input devices to provide inputs into the system, and the system processes that information to develop output commands for the robotic manipulator in order to move the instruments and, as appropriate, operate the instrument end effectors. The user interface may be one that allows the surgeon to select motion scaling factors as well as to clutch and reposition the handles to a more comfortable position. In some cases, the surgeon may desire a fine scaling motion, while in others he may prefer larger motion, relative to the movement of the user interface.

The concept described in this application involves the use of the haptic input device to create virtual tactile boundaries that define the useful ergonomic workspace of the input device to enable more automatic and natural clutch activation. When the user, while moving the user input, reaches these boundaries, which may be near the end of range of motion of the device and/or that the user's pose is no longer comfortable or effective position, a number of different haptic features could be used to assist the user with clutching. These include the following:

1. Virtual Walls with Force/Motion Gesture Clutch

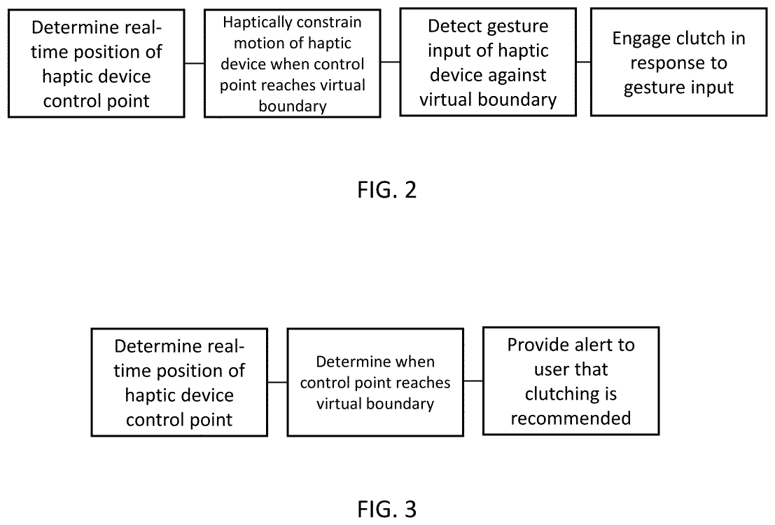

With this feature, depicted in FIG. 2, haptic constraints are created to act as virtual walls at the edges of the useful workspace of the device. In the control system, the real time position and orientation of the control point near the input handle is monitored, such as using input from position sensors associated with the user input device. When the position of the control is determined to be outside of the predetermined defined virtual boundaries (edges of the walls) stored in the system's memory, the control system will generate a force/torque at the control point in the opposite direction. In one possible implementation, the magnitude of this correcting force/torque is proportional to the difference between the position of the control point and the position of the wall. This distance term is then multiplied by a constant value to set the magnitude of force that is applied to the handle by the motors. In this example, the virtual walls would feel like springs of spring rate equal to the constant value described previously, trying to push the user back into the workspace.

To cause the system to clutch, rather than using the conventional approach of depressing the foot pedal of the work station or engaging other switch/input, the user could move the haptic user input in the direction of this virtual boundary, to “press” against this virtual boundary. This action serves as input to the control system that the user would like to disable the output motion of the manipulator. Examples of gestures that might be used to function as a haptic clutch include, without limitation:

direction of force/torque

frequency of force/torque

number of instances of force/torque over a time period

duration of application of force/torque

direction and/or distance of displacement of the control point

In one exemplary embodiment, the user moves the user input device twice in succession, to press twice against the virtual wall, or moves the user input device and holds against the wall for a certain time, or presses hard/far enough into the wall to cause the system to recognize the input as an instruction to clutch. The choice of which gestures to use as a haptic clutch could be made by evaluating user preferences and programming the system to recognize those gestures as instructions to perform clutching.

2. Virtual Boundary with Haptic Alert

In an alternative configuration depicted in FIG. 3, a haptic or vibratory alert is used to indicate to the user that he/she is in a position in which clutching and repositioning is recommended. This mode would allow the user to keep operating up to the limits of the device but provides an alert to remind the user to clutch rather than continuing to operate in a poor ergonomic position. This might be particularly useful with surgeons who are new to using robotic surgical systems. For those surgeons, clutching is a new feature to learn, and so a regular reminder to clutch and re-center for comfort could be beneficial. This concept would provide those reminders near the limits of the input device, similar to a warning track in the outfield of a baseball field.

3. Virtual Boundary with Visual or Audible Alert

This concept is identical in purpose and function to concept (2) except that a visual or audible alert on the monitor displaying the surgical field or from the user interface console is provided instead of a haptic alert.

4. Virtual Boundary with Auto-Clutch

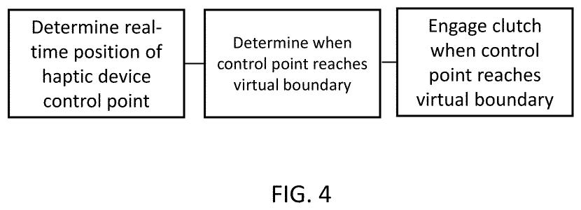

In this final option, depicted in FIG. 4, the system automatically clutches to disable output motion when the virtual boundary is reached. For a more experienced user, this type of control mode could be very efficient as he/she would know from experience that he/she is near the workspace limits and expect to be auto-clutched soon. Upon auto-clutching, the user could quickly re-center his/her hands and continue operating. A haptic, visual, or audible alert may also be used to immediately notify the surgeon that he/she has been clutched out at the moment that the virtual boundary is reached.

Once the control system deactivates the output motion input motion relationship, the system can assist with repositioning the user's hands and re-enabling the control of the output device in any of the following ways:

### 1. Autonomous Motion to Optimal Start Position

Once the system is clutched out via one of the methods discussed above, the manipulator autonomously moves the handle into the optimal start position. The user can then resume the procedure and clutch back in simply by moving the handles. Alternatively, a haptic constraint can be applied so that the user needs to apply a force greater than some threshold for the system to clutch back in and enable control of the output device.

2. Virtual Wall at Optimal Start Position with Force/Motion Gesture Clutch (1+ Planes)

Once the system is clutched out via one of the methods discussed above, the user can reposition his/her hand back to the optimal starting position. At this optimal start position, the user will feel the virtual walls being created by the control system and he/she can use force/position gestures (as described previously) to clutch back in. The virtual walls may be 1 or more virtual planes enabling the user some flexibility in start pose. If 1 plane is used, it may allow the user to clutch back in at any height or depth but only at the lateral position at which the virtual plane is created. The choice of position and orientation of the virtual wall can be made by the control system based on which virtual boundary was crossed when clutching out. For example, if the maximum right side limit was hit, the system may create a haptic wall positioned at the optimal start position such that the user will feel the wall after moving the handle back to the left.

### 3. Haptic, Visual, or Audible Alert at Optimal Start Position

This option is identical to the previous option (2) except that an alert is provided to the user via haptic vibration, visual alert, and/or audible alert once the virtual boundary is crossed that defines the optimal start pose. This will tell the user that this is a good ergonomic position at which to clutch back in.

The disclosed concepts provide several advantages over existing technology. Current robotic systems use buttons, pedals, surgeon presence sensors, or other switches to clutch in and out for hand repositioning. This can, at times, be cumbersome, especially for new users. Such prior methods also require the use of a finger, foot, etc to actuate the clutch which prevents them from being used for other purposes. Furthermore, those steps take time, require training, and leave room for ergonomic and usability improvements. Clutching is a critical advantage that robotic surgery offers over manual surgical methods as it enables improved ergonomics, strength, and confidence for surgeons performing the procedures. The disclosed methods of using virtual boundaries, virtual walls, and force/motion gesture clutching to clutch in and out of control of the output device enables users to maintain hands on the input device, use hands, fingers, and feet for other tasks, reminds users to clutch when outside of ergonomic limits, and accelerates the clutching process by increasing ease of use and increasing surgeon robot collaboration. These methods should be much more natural to a user and should help increase effective utilization of robotic clutching.

I believe that the use of virtual boundaries and/or virtual walls to provide haptic, audible, or visual alerts to the user to encourage ergonomic adjustment and suggest the use of clutching is novel for surgical robotics. Automatic clutching at virtual boundaries is also novel to my understanding. I also believe that the use of virtual walls with haptic force/motion gesture clutching is entirely new in our field. As far as I know, all of the functionality described in the technical details section is novel.

All prior patents and applications referred to herein, including for purposes of priority, are incorporated herein by reference.

We claim:

1. A robot-assisted surgical system comprising:
a robotic manipulator configured for robotic positioning of a surgical instrument in a body cavity,
at least one haptic user input device moveable by a user,
at least one processor and at least one memory, the at least one memory storing instructions executable by said at least one processor to:
define virtual boundaries in a workspace of the user input device, the virtual boundaries defined based on range limits or user ergonomic limits of the user input device;
receive user input in response to movement of the input device by a user;
cause the manipulator to move the first surgical instrument in response to the user input,
casing an alert to the user to be generated in response to movement of the user input device in proximity of the virtual boundary, the alert alerting the user to clutch and reposition the user input device.

2. The system of claim 1, wherein the user input device is a haptic input device, and wherein the alert is an activation of actuators of the haptic input device.

3. The system of claim 2, wherein the alert is an activation of the actuators to cause the user input device to push against the user in a direction opposed to the direction of movement of the input device.

4. The system of claim 1, wherein the alert causes vibration of the user input.

5. The system of claim 1, wherein the alert is an auditory alert.

6. The system of claim 1, wherein the alert is a visual alert displayed on a display observable by the user.

7. The system of claim 2, wherein the memory stores instructions executable by the processor to recognize predetermined input from the user input as a clutch instruction, and to suspend the input/output relationship between the user input and the manipulator in response to the clutch instruction.

8. The system of claim 7, wherein the predetermined input is selected from any of the following sensed at the user input device:
direction of force/torque
frequency of force/torque
number of instances of force/torque over a time period
duration of application of force/torque
direction and/or distance of displacement of the control point

9. The system of claim 1, wherein the memory stores instructions executable by the processor to suspend the input/output relationship between the user input and the manipulator in response to movement of the user input device in proximity of the virtual boundary.

10. The system of claim 7, wherein the memory stores instructions executable by the processor to cause actuators of the input device to move the user input device to a predetermined starting position in response to suspension of the input/output relationship between the user input and the manipulator.

11. The system of claim 7, wherein the memory stores instructions executable by the processor to, in response to user repositioning of the user input device during suspension of the input/output relationship between the user input and the manipulator, defining second virtual boundaries in the workspace of the user input device, and response to user interaction with the virtual boundaries using the user input device, re-engaging the input/output relationship between the user input and the manipulator.

12. The system of claim 11, wherein the user interaction is selected from any of the following sensed at the user input device:
direction of force/torque
frequency of force/torque
number of instances of force/torque over a time period
duration of application of force/torque
direction and/or distance of displacement of the control point.

13. The system of claim 7, wherein the memory stores instructions executable by the processor to, in response to user repositioning of the user input device during suspension of the input/output relationship between the user input and the manipulator, causing a haptic, auditory or visual alert to the user indicating to the user in response to a determination that the user input device has been re-positioned to a suitable starting position.

14. The system of claim 9, wherein the memory stores instructions executable by the processor to, in response to user repositioning of the user input device during suspension of the input/output relationship between the user input and the manipulator, causing a haptic, auditory or visual alert to the user indicating to the user in response to a determination that the user input device has been re-positioned to a suitable starting position.

15. The system of claim 9, wherein the memory stores instructions executable by the processor to cause actuators of the input device to move the user input device to a predetermined starting position in response to suspension of the input/output relationship between the user input and the manipulator.

16. The system of claim 9, wherein the memory stores instructions executable by the processor to, in response to user repositioning of the user input device during suspension of the input/output relationship between the user input and the manipulator, defining second virtual boundaries in the workspace of the user input device, and response to user interaction with the virtual boundaries using the user input device, re-engaging the input/output relationship between the user input and the manipulator.
